(19) United States
(12) Patent Application Publication
(10) Pub. No.: US 2012/0123355 A1
(43) Pub. Date: May 17, 2012

(54) ERODIBLE EMBOLIZATION MATERIAL

(75) Inventors: Dennis J. Delap, Bloomington, IN (US); Cleve S. Koehler, Ellettsville, IN (US)

(73) Assignee: Cook Medical Technologied, Bloomington, IN (US)

(21) Appl. No.: 13/387,149

(22) PCT Filed: Jul. 30, 2010
(86) PCT No.: PCT/US10/43818

§ 371 Date: Jan. 26, 2012

(30) Foreign Application Priority Data
Jul. 30, 2009 (US) 61229939

(51) Int. Cl. A61M 5/00; A61K 9/00

(52) U.S. Cl. 604/272; 424/400

(57) ABSTRACT

An embolization agent for use with a therapeutic drug solution for treating diffuse diseases in a targeted tissue vessel of a patient and a process thereof is presented. This embolization agent comprises an inner core of a fast dispersing material having a diameter less than a predetermined size for the opening in the targeted tissue vessel; and an outer sphere of an embolization material encompassing the inner core and having an initial diameter to occlude the initial opening in the targeted tissue vessel. During the treatment of the disease, the outer sphere of embolization material erodes at a predetermined rate, enhancing penetration of the embolization agent into the opening in the targeted tissue vessel. The inner core disperses into the targeted tissue vessel after becoming exposed by the erosion of the embolization material of the outer sphere.

### CROSS REFERENCE TO RELATED APPLICATIONS

This application claims the benefit of priority to U.S. Provisional Application Ser. No. 61/229,939 filed Jul. 30, 2009, the entire contents of which are hereby incorporated herein by reference.

### BACKGROUND OF THE INVENTION

1. Field of the Invention

This invention relates to embolization, as well as to the treatment of diffuse diseases and localized tumor destruction.

2. Background

Diffuse diseases (e.g., malignant metastatic melanoma, etc.) that can be present in various body systems, such as the lymph nodes, are difficult to treat using conventional drug therapy or chemotherapy techniques. Conventional techniques typically include the surgical resection or removal of the affected tissue. These techniques can be ineffective due to the disease being present in adjacent tissue or other organs. In this case, it is possible that the disease may reemerge after surgical procedures are deemed initially successful.

Chemo-embolization is a technique that has been successful in treating well-defined cancerous tumors. This technique involves the use of an embolization material to deliver a concentrated dosage of a chemotherapy drug to a tumor with subsequent surgical removal of the resulting malignant tumor. The advantage of this technique lies in the fact that it provides a synergistic effect of embolization and local chemotherapy. The chemo-embolization materials used in this technique are considered “permanent” in that activity is extended for a substantial length of time, thereby, ensuring tissue morbidity. For example, Gelfoam™ (Pfizer, N.Y.) is known to have a two-week effective embolization time.

It would be advantageous to the medical industry to utilize an embolization technique to treat diffuse diseases in addition to well defined tumors. Thus, there is a need in the industry to develop a material and devise a method for applying a chemo-embolization technique for use with tissues that have a minimal amount or even an unquantifiable amount of a disease.

### SUMMARY

The present invention generally provides an embolization agent for use with a therapeutic drug for treating diffuse diseases in a targeted tissue vessel of a patient or for localized tumor destruction. In one embodiment, the embolization agent comprises an inner core of a fast dispersing material having a diameter less than a predetermined size for the opening in the targeted tissue vessel and an outer sphere of an embolization material encompassing the inner core. The outer sphere has an initial diameter that occludes the initial opening in the targeted tissue vessel. During the treatment of the disease, the outer sphere of embolization material erodes at a predetermined rate, enhancing penetration of the embolization agent into the opening of the targeted tissue vessel. The inner core of fast dispersing material disperses into the targeted tissue vessel after becoming exposed by the erosion of the embolization material of the outer sphere.

In one embodiment, the embolization agent is suspended within a therapeutic drug solution and delivered with the drug solution to the targeted tissue vessels in the patient. In another embodiment, a therapeutic drug solution is delivered to the targeted tissue vessel in the patient immediately following delivery of the embolization agent to the targeted tissue vessel. The therapeutic drug solution may be any chemotherapy drug known to one skilled in the art of cancer treatment. One example of such a chemotherapy drug is paclitaxel.

In one embodiment of the present invention, the targeted tissue vessels may have an unquantifiable amount of disease. These targeted tissue vessels may be fluid-transfer vessels, including but not limited to vascular or lymphatic vessels. Although these vessels may be located elsewhere, they preferably are located in either liver or kidney tissue.

In another embodiment of the present invention, the outer sphere of embolization material erodes at a predetermined rate that is predictable and controllable. This predetermined rate of erosion results in substantial erosion of the outer sphere in a timeframe of about 30 seconds to 24 hours. In addition, the outer sphere of embolization material erodes at a similar rate when in contact with the vessel walls or when in contact with a fluid located in the vessels. After erosion, any existing fragments of the embolization material are smaller than the size expected to cause a stroke or any other type of complication in the patient.

The diameter of the outer sphere is typically less than about 100 micrometers, while the diameter of the inner core is less than about 40 micrometers. The embolization material of the outer sphere may be selected from any embolization material known to one skilled in the art and the fast dispersing material of the inner core may be selected from any fast dispersing material known to one skilled in the art that does not adversely affect the body.

It is another objective of the present invention to provide a procedure for treating diffuse diseases in targeted tissue vessels of a patient. This procedure comprises the steps of introducing to a targeted tissue vessel, an embolization agent and a therapeutic drug solution. The embolization agent includes an inner core of a first fast dispersing material and an outer sphere of a second erodible embolization material. The embolization agent occludes the targeted tissue vessel in the initial portions thereof. The procedure further includes eroding the outer sphere of embolization material at a predetermined rate to enhance penetration into the targeted tissue vessel and to expose the inner core; and dispersing the inner core of the first material into the targeted tissue vessel when the inner core becomes substantially exposed.

In another embodiment of the present invention, a delivery system for delivering a treatment for diffuse diseases to a targeted tissue vessel in a patient's body includes an introducer apparatus and an embolization agent having an inner core of a first fast dispersing material and an outer sphere of a second embolization material that encompasses the inner core. In this embodiment, the inner core has an initial first diameter less than the predetermined size of the opening in the targeted tissue vessel and the outer sphere has an initial second diameter that occludes the initial opening in the targeted tissue vessel. The outer sphere of embolization material erodes at a predetermined rate, enhancing penetration of the embolization agent into the opening in the targeted tissue vessel and allows the inner core of the first material to disperse into the targeted tissue vessel after becoming substantially exposed by the erosion of the outer sphere.

In one embodiment, the introducer apparatus is a fluid delivery apparatus, in which the embolization agent is suspended within a solution and the fluid delivery apparatus delivers the premixed solution into the targeted tissue vessel of the patient. The solution may be a therapeutic drug. Alternatively, the solution may be saline, in which case a therapeutic drug chases the embolization agent-saline solution.

In another embodiment, the introducer apparatus is a wire-catheter arrangement including a hollow needle to pierce the patient's body, a wire guide for insertion into the hollow needle with percutaneous advancement into the patient's body to the desired targeted tissue vessel allowing for the subsequent removal of the hollow needle, a catheter having a proximal and distal end advanced along the wire guide to the desired targeted tissue vessel allowing removal of the wire guide; and a pusher member to advance the embolization agent from the proximal end of the catheter through the distal end of the catheter into the targeted tissue vessel. In this embodiment, the embolization agent is chased with a therapeutic drug solution during delivery to the targeted tissue vessel.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a cross-sectional view of an erodible embolization agent according to one embodiment of the present invention;

FIGS. 2A-D are cross-sectional views depicting the erosion of the outer sphere of the embolization agent shown in FIG. 1 that allows for enhanced penetration of the material into the targeted vessel; and

FIG. 3 is a schematic view of a method for treating a patient with an embolization agent in accordance with one example of the present invention.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

A chemo-embolization agent for treating diffuse diseases in a targeted tissue vessel of a patient is disclosed in U.S. provisional application Ser. No. 61/093,505, filed on Sep. 2, 2008, by Delap, entitled ERODIBLE EMBOLIZATION MATERIAL, the entire contents of which are incorporated herein by reference. The chemo-embolization agent disclosed in the 61/093,505 application comprises an inner core of a chemotherapy drug and an outer sphere of an embolization material encompassing the inner core. During treatment of the disease, the outer sphere of embolization material erodes at a predetermined rate, enhancing penetration of the chemo-embolization agent into the opening of the targeted tissue vessel. The inner core of chemotherapy drug disperses into the targeted tissue vessel after becoming exposed by the erosion of the embolization material of the outer sphere. The outer sphere of embolization material may further comprise a therapeutic material, such as a chemotherapy drug.

An object of the chemo-embolization agent disclosed in the 61/093,505 application, which includes a chemotherapy drug embedded within the inner core, and possibly within the outer sphere, is the treatment of diseased tissue with a high concentration of drug in a targeted area of a patient's vessel. The chemo-embolization agent occludes the targeted vessel and as the chemo-embolization agent erodes, it disperses the chemotherapy drug embedded within into the occluded vessel. While the chemo-embolization agent disclosed in the 61/093,505 application provides effective treatment of diseased tissue, embedding the chemotherapy drug within the embolization agent is not necessary. Embodiments of the present invention generally provide an embolization agent separate from, but for use with, a therapeutic drug, such as a chemotherapy drug, for treating diffuse diseases in a targeted tissue vessel of a patient. Embodiments of the embolization agent of the present invention may be suspended within a therapeutic drug solution and delivered to a targeted tissue vessel, or the embolization agent may be delivered to a targeted tissue vessel and chased with a therapeutic drug solution.

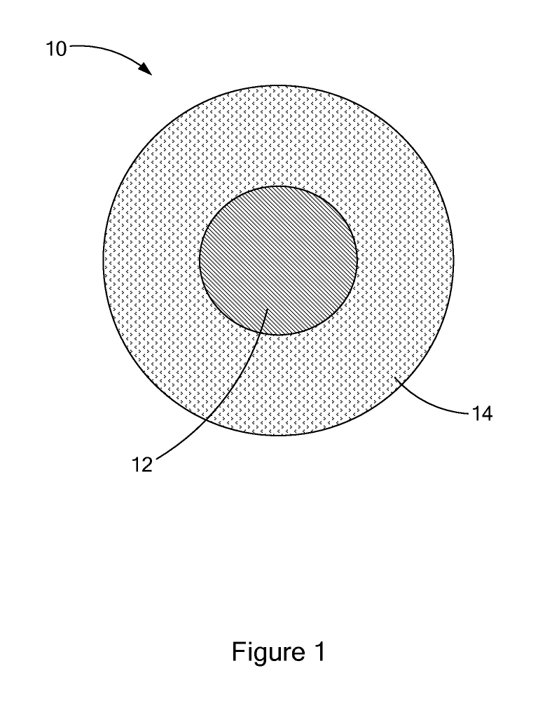

Referring to FIG. 1, the embolization agent 10 preferably comprises an inner core 12 of a fast dispersing material that has a diameter less than a predetermined size for the initial opening in a targeted tissue vessel 15 and an outer sphere 14 of an embolization material that encompasses the inner core 12 and has an initial diameter that will occlude the initial opening 20 in the targeted tissue vessel 15. The outer sphere 14 may be comprised of a single layer or multiple layers that are different or similar in composition. In this embodiment, the outer sphere 14 of embolization material erodes at a predetermined rate, which enhances penetration of the embolization agent 10 into the opening of the targeted tissue vessel 15. Upon becoming exposed by the erosion of the embolization material of the outer sphere 14, the inner core 12 preferably disperses rapidly into the targeted tissue vessel 15 within a matter of seconds to hours without obstructing subsequent blood flow.

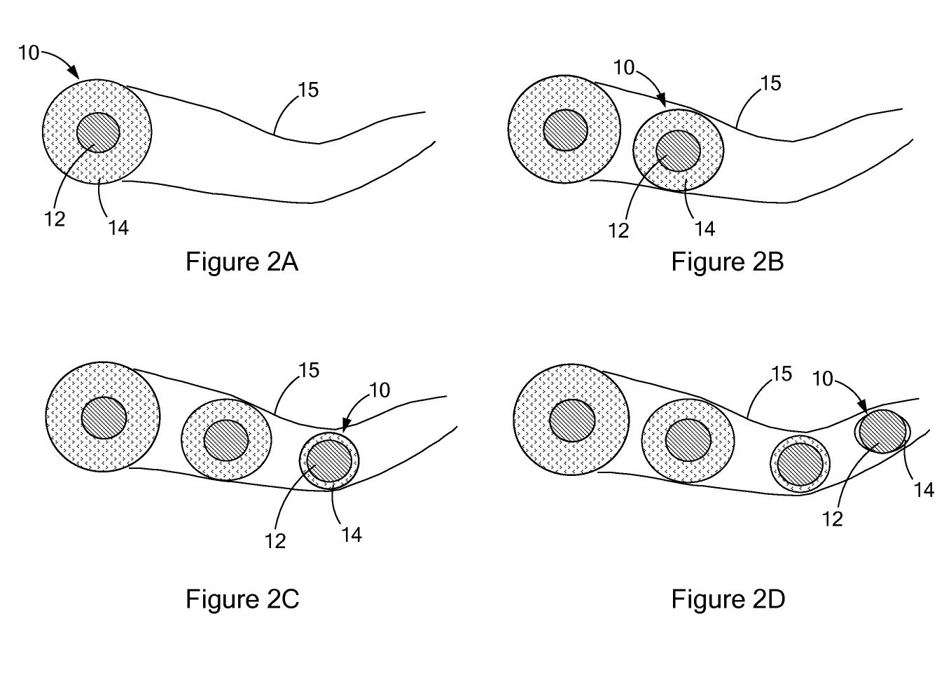

Referring to FIGS. 2A-D, during the treatment of the disease, the outer sphere 14 of embolization material preferably erodes at a predetermined rate, enhancing penetration of the embolization agent 10 further into the opening in the targeted tissue vessel 15 as depicted in FIGS. 2A, 2B, and 2C. In this embodiment, the inner core 12 of embolization material disperses into the targeted tissue vessel 15 after becoming exposed by substantial erosion of the embolization material of the outer sphere 14, as depicted in FIG. 2D.

The tissue vessels 15 targeted by the use of the embolization agent 10 may have a minimal or unquantifiable amount of disease. These targeted tissue vessels 15 may be fluid-transfer vessels, including but not limited to vascular or lymphatic vessels. The embolization agent 10 preferably is used to treat vessels 15 located in either the tissue of the liver or kidney, however, such treatment may be used with vessels 15 located elsewhere.

In this embodiment, the outer sphere 14 of embolization material erodes at a predetermined rate that is predictable and controllable. By erosion, the embolization material may become disassembled, digested, or metabolized into smaller or dispersed molecules through the action of the surrounding environment present in the targeted tissues vessels. Eroded embolization material may form fragments that will continue to erode. These erodible fragments of embolization material are smaller than the size expected to cause a stroke or any other type of complication in the patient.

This predetermined rate of erosion results in substantial erosion of the outer sphere 14 in a timeframe of about 30 seconds to 24 hours. Upon substantial erosion of the outer sphere 14, the inner core 12 is able to disperse into the targeted tissue vessel 15. The properties of the embolization material may be adjusted to be suitable for use with vascular, lymphatic, or other fluid-transfer vessels. In addition, the outer sphere 14 of embolization material may erode at a similar rate when in contact with the vessel 15 walls or when in contact with a fluid located in the vessels 15. Maintaining a similar rate of erosion during treatment assists in keeping the embolization effect substantially constant as the embolization agent 10 progresses through smaller and smaller vessels.

In this embodiment, the diameter of the outer sphere 14 of embolization material is typically less than about 100 micrometers, while the diameter of the inner core 12 of fast dispersing material is preferably less than about 40 micrometers. In comparison, liver tumor vasculature vessels have an opening on the order of about 40 to 80 micrometers, while normal healthy liver tissue has vascular vessels with openings between about 7 to 40 micrometers. Thus, the outer sphere 14 has a diameter that is slightly larger than the initial opening in the targeted vessels, while the inner core 12 has a diameter either the same or slightly smaller than the expected size of the opening in the targeted vessels. The exact size of the openings associated with other vascular or lymphatic tissue vessels may vary from the above description. Such variation is contemplated to be within the scope of the present invention.

The embolization material of the outer sphere 14 may be any embolization material known to one skilled in the art including but not limited to polyvinyl alcohol, tris-acryl gelatin microspheres, gelatin sponge microfibrillar collagen, ethoiodized oil, autologous materials, polyvinyl alcohol, celluloses, polyacrylic acids, polyacylamides, and alginates or mixtures thereof. The embolization material may be multi-component in that it can be comprised of a polymer solution and a gelling precursor.

The material of the inner core 12 may be any fast dissolving solid material that does not adversely affect the body known to one skilled in the art including but not limited to sugar, sucrose, lactose, fructose, or any fast eroding polymer known in the art. The inner core may be ionically or covalently bonded to the embolization material of the outer sphere 14 or held in place by entanglement, van der Waal forces, hydrogen bonding, or any other means known to one skilled in the art of particle encapsulation.

In one embodiment of the present invention, the embolization agent 10 may be spherical or ellipsoidal in shape. Non-spherical or non-ellipsoidal shapes are similarly allowable, however, some sort of symmetry is preferred to obtain a uniform and predictable erosion rate. Since the embolization agent 10 will be disposed within blood vessels having a circular cross-section, the symmetry is preferably at least radial to obtain consistent embolization time intervals. For example, the embolization agent 10 may take the shape of a sphere or ellipsoid with two axes having a length approximately equal to one another and a third axis greater than the other two, i.e., football-shaped without the pointed ends. Accordingly, the term “outer sphere,” as used herein, may be used interchangeably with “outer layer” and includes any spherical or ellipsoidal shape having radial symmetry. Such shaping of the embolization agent 10 may occur during or after formation of the embolization agent 10 using any process known to one skilled in the art, such as molding, compression, or agglomeration, among others.

In another embodiment, the inner core 12, outer sphere 14, or both may further comprise a radioopaque material. The radiopaque material may be discrete particles or a coating. The radiopaque material may be a polymer, ceramic, or a noble metal. Examples of noble metals include gold, platinum, iridium, palladium, or rhodium, or a mixture thereof. The radiopaque material provides enhanced fluoroscopy to more easily identify the location of the embolization agent 10 during delivery,

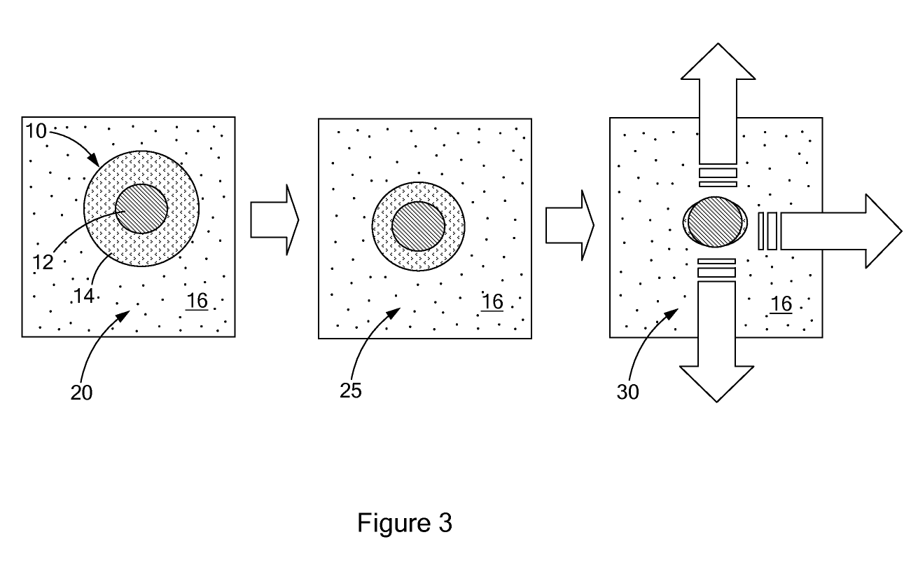

It is another objective of the present invention to provide a method for treating diffuse diseases in targeted tissue vessels 15 of a patient. Referring to FIG. 3, this method comprises delivering (20) an embolization agent 10 to the targeted tissue vessel 15 so that the targeted tissue vessel 15 is occluded in the initial portions thereof. This may be accomplished by suspending the embolization agent 10 in solution and introducing the solution premixed with the embolization agent into the patient through the use of a fluid delivery apparatus. In one embodiment, the embolization agent 10 may be suspended in a saline solution that is delivered through the fluid delivery apparatus and then chased with a therapeutic drug solution, such as any chemotherapy drug known in the art. Alternatively, a therapeutic drug solution 16 may serve as the carrier for the embolization agent 10, in which case the embolization agent 10 is suspended within the therapeutic drug solution 16 and the fluid delivery apparatus dispenses the therapeutic drug solution premixed with the embolization agent 10 to the targeted tissue vessels within the patient, as best illustrated in FIG. 3.

In another embodiment, the embolization agent 10 may be introduced into the patient by using an introducer apparatus, such as a wire-catheter arrangement well known to one skilled in the art of medicine, and chasing the embolization agent 10 with a therapeutic drug solution, such as any chemotherapy drug known in the art. In this embodiment, a wire-catheter arrangement includes using a hollow needle to pierce the patient's skin and enter the body tissue at an angle with respect thereto. A wire guide is then inserted into the hollow needle and is advanced percutaneously into the body tissue to the desired position for delivery of the embolization agent 10 to the targeted tissue vessels 15. The hollow needle is then pulled in a backward direction so as to be removed from the body tissue and from contact with the wire guide. Next, a catheter is advanced along the wire guide to the desired position.

The catheter has a distal end through which the embolization agent 10 is delivered into the targeted tissue vessel 15. The catheter is preferably made of a soft, flexible material such as silicon or any other suitable material. Generally, the catheter also has a proximal end and a plastic adaptor to receive the embolization agent 10. The diameter of the catheter is based upon the size of the body tissue into which the catheter is inserted and the amount of embolization agent 10 to be delivered. The inner diameter of the catheter is larger than the diameter of the outer sphere 14 of the embolization agent 10 to allow delivery of the agent to the targeted tissue vessel 15.

In use, the embolization agent 10 may be placed in the catheter prior to insertion into the body tissue. The embolization agent 10 is then guided through the catheter preferably from the proximal end and distally beyond the distal end of the catheter to a location within the vascular tissue near or at the targeted tissue vessel 15.

The delivery assembly may further include a polytetrafluoroethylene (PTFE) introducer sheath to assist the percutaneous introduction of the wire guide and the catheter in the body tissue. Of course, any other suitable material may be used for the sheath. The introducer sheath facilitates inserting the catheter percutaneously to a desired location in the body tissue and provides stability to the catheter at the desired location in the body tissue. For example, as the introducer sheath is held stationary within the vascular tissue of the liver, it adds stability to the catheter, as the catheter is advanced through the introducer sheath to a desired location in the vascular tissue.

When the distal end of the catheter is at a location near the targeted tissue vessel 15, the embolization agent 10 is inserted into the catheter and is advanced coaxially through the catheter for delivery through the distal end of the catheter. In this configuration, a shaft or other pusher member can be used to advance or push the embolization agent 10 from the proximal end through the catheter and exiting the distal end of the catheter at or near the targeted tissue vessel 15. In this embodiment, the embolization agent 10 is chased with a therapeutic drug solution by means of a fluid injection apparatus well known to one of skill in the art.

The method further comprises eroding (25) the outer sphere 14 of embolization material at a predetermined rate to enhance penetration into the targeted tissue vessel 15 and to expose the inner core 12. The step of eroding (25) the outer sphere 14 of embolization material occurs at a similar rate whether the material is in contact with the vessels' walls or with the fluids present in the vessels.

The method further comprises dispersing (30) the inner core 12 into the targeted tissue vessel 15 when the inner core 12 becomes substantially exposed.

The effective concentration or dosage of the embolization agent and therapeutic drug solution can be determined and varied by the physician treating the patient. Such a decision should be based on the nature, location, and severity of the condition to be treated and the method selected to administer the agent. Any means known to one skilled in the art may be used to introduce an acceptable dosage of the embolization agent and therapeutic drug solution to the targeted tissue. An appropriate concentration of embolization agent and therapeutic drug solution may be used for localized tumor destruction, as well as for treating diffuse diseases.

The outer sphere 14 of embolization material may further comprise anti-thrombogenic agents and agents that promote or inhibit clotting.

Rather than embedding a therapeutic drug within the inner core 12 and/or outer sphere 14 of the embolization agent 10 of the present invention, the embolization agent 10 is either suspended within a therapeutic drug solution, or chased with a therapeutic drug solution, and delivered to a targeted vessel 15 within a patient. The embolization agent 10 of the present invention provides an outer sphere 14 which erodes at a predetermined rate in order to get the blood supply cutoff and highly concentrated drug exposure in a desired time frame and an inner core 12 of material that disperses very quickly to avoid risk of stroke caused by potential fragments leaving the area and being recycled around the body.

A person skilled in the art will recognize from the previous description that modifications and changes can be made to the present disclosure without departing from the scope of the disclosure as defined in the following claims.

What is claimed is:

1. An embolic agent configured for delivery to targeted tissue vessels in a patient, the agent comprising:
an inner core of a first material, the inner core having a diameter less than a predetermined size for the opening in the targeted tissue vessel; and
an outer sphere of a second embolization material encompassing the inner core and having an initial diameter to occlude the initial opening in the targeted tissue vessel;
wherein the outer sphere of the second embolization material erodes at a predetermined rate, enhancing penetration of the embolic agent into the opening in the targeted tissue vessel;
wherein the inner core of the first material disperses into the targeted tissue vessel after becoming exposed by the erosion of the second embolization material of the outer sphere.

2. The embolic agent of claim 1, wherein the predetermined rate at which the outer sphere of the second embolizaton material erodes is predictable and controllable.

3. The embolic agent of claim 1, wherein the predetermined rate of erosion for the outer sphere of the second embolization material results in substantial erosion of the outer sphere in a timeframe of about 30 seconds to 24 hours.

4. The embolic agent of claim 2, wherein the outer sphere of the second embolization material erodes at a similar rate when in contact with the vessel walls or when in contact with a fluid located in the vessels.

5. The embolic agent of claim 3, wherein after erosion, any existing fragments of the second embolization material are smaller than the size expected to cause a stroke or other type of complication in the patient.

6. The embolic agent of claim 1, wherein the diameter of the outer sphere of the second embolization material is less than about 100 micrometers and the diameter of the inner core of the first material is less than about 40 micrometers.

7. The embolic agent of claim 1, wherein the first material of the inner core is one selected from the group of sugar, sucrose, lactose, fructose, and fast eroding polymers or mixtures thereof.

8. The embolic agent of claim 1, wherein the second embolization material of the outer sphere is one selected from the group of polyvinyl alcohol, tris-acryl gelatin microspheres, gelatin sponge microfibrillar collagen, ethoiodized oil, autologous materials, polyvinyl alcohol, celluloses, polyacrylic acids, polyacylamides, and alginates or mixtures thereof.

9. The embolic agent of claim 1, wherein the embolic agent is suspended within a therapeutic drug solution and the suspension of the embolic agent and the therapeutic drug solution is delivered to the targeted tissue vessels in the patient.

10. The embolic agent of claim 9, wherein the therapeutic drug solution is a chemotherapy drug selected from the group of alkylating agents, antimetabolites, anthracyclines, plant alkaloids, antitumor antibiotics, taxanes, platinum derivatives, paclitaxel, L-asparaginase, hydroxyurea, thalidomide, and dactinomycin, or mixtures thereof.

11. The embolic agent of claim 1, wherein the embolic agent is chased with a therapeutic drug solution during delivery to the targeted tissue vessels in the patient, wherein the drug solution is a chemotherapy drug selected from the group of alkylating agents, antimetabolites, anthracyclines, plant alkaloids, antitumor antibiotics, taxanes, platinum derivatives, paclitaxel, L-asparaginase, hydroxyurea, thalidomide, and dactinomycin, or mixtures thereof.

12. The embolic agent of claim 1, wherein at least one of the inner core and outer sphere further comprises a radiopaque material.

13. A delivery system for delivering a treatment for diffuse diseases to a targeted tissue vessel in a patient's body, the delivery system comprising:
an introducer apparatus; and
an embolization agent having an inner core of a first material and an outer sphere of a second embolization material that encompasses the inner core, the inner core having an initial first diameter less than the predetermined size of the opening in the targeted tissue vessel, the outer sphere having an initial second diameter that occludes the initial opening in the targeted tissue vessel, wherein the outer sphere of the second embolization material erodes at a predetermined rate, enhancing penetration of the embolization agent into the opening in the targeted tissue vessel and allowing the inner core of the first material to disperse into the targeted tissue vessel after becoming substantially exposed by the erosion of the outer sphere.

14. The delivery system of claim 13, wherein the introducer apparatus is a fluid delivery apparatus, wherein the fluid delivery apparatus introduces into the targeted tissue vessel of the patient a solution premixed with the embolization agent, wherein the solution is one of saline and a therapeutic drug solution.

15. The delivery system of claim 13, wherein the introducer apparatus is a wire-catheter arrangement comprising:
a hollow needle to pierce the patient's body;
a wire guide for insertion into the hollow needle with percutaneous advancement into the patient's body to the desired targeted tissue vessel allowing for the subsequent removal of the hollow needle;
a catheter having a proximal and distal end advanced along the wire guide to the desired targeted tissue vessel allowing for removal of the wire guide; and
a pusher member to advance the embolization agent from the proximal end of the catheter through the distal end of the catheter into the targeted tissue vessel, wherein the embolization agent is chased with a therapeutic drug solution during delivery to the targeted tissue vessel in the patient.
